(19) United States
(12) Patent Application Publication
(10) Pub. No.: US 2019/0112635 A1
(43) Pub. Date: Apr. 18, 2019

(54) SYSTEMS AND METHODS FOR HIGH THROUGHPUT AND PARALLEL CHROMATIN IMMUNOPRECIPITATION ASSAYS

(71) Applicants: Chang Lu, Blacksburg, VA (US); Travis Murphy, Blacksburg, VA (US)

(72) Inventors: Chang Lu, Blacksburg, VA (US); Travis Murphy, Blacksburg, VA (US)

(73) Assignee: Virginia Polytechnic Institute and State University, Blacksburg, VA (US)

(21) Appl. No.: 16/163,702

(22) Filed: Oct. 18, 2018

Related U.S. Application Data
(60) Provisional application No. 62/573,957, filed on Oct. 18, 2017.

(51) Int. Cl. C12Q 1/6804; C12Q 1/6874; B01J 20/32

(52) U.S. Cl. CPC C12Q 1/6804; B01J 20/321; C12Q 1/6874

(57) ABSTRACT

LIFE-ChIP-seq (Low-Input Fluidized-bed Enabled Chromatin Immunoprecipitation followed by sequencing), an automated and high-throughput microfluidic platform capable of running multiple sets of ChIP assays in as little as 1 hour with as few as 50 cells per assay. This technology enables testing of a large number of samples and replicates with low-abundance primary samples in the context of precision medicine.

### CROSS-REFERENCE TO RELATED APPLICATIONS

This application claims the benefit of U.S. provisional patent application No. 62/573,957, filed Oct. 18, 2017, the complete contents of which are herein incorporated by reference.

### STATEMENT OF FEDERALLY SPONSORED RESEARCH AND DEVELOPMENT

This invention was made with government support under grant numbers EB017235 and CA214176 awarded by the National Institutes of Health. The government has certain rights in the invention.

### FIELD OF THE INVENTION

The invention is generally directed to low-input chromatin immunoprecipitation followed by sequencing (ChIP-seq) technologies, in particular low-input ChIP-seq technologies allowing high-throughput and simultaneous processing of multiple samples. Exemplary applications include automated and parallel analysis of histone modifications.

### BACKGROUND OF THE INVENTION

The primary tool utilized for examining histone modifications is chromatin immunoprecipitation (ChIP), which applies immunoassay to capture chromatin fragments by targeting the specific type of modified histone bound to them. These chromatin fragments yield DNA fragments that can be sequenced (i.e. ChIP-seq) and provide a genome-wide map of histone binding. ChIP-seq can also be used to profile bindings of molecules such as RNA polymerases and transcription factors to the genome.

There are a couple of critical limitations associated with conventional ChIP-seq assays. First, they typically require a large number of cells (˜10⁷-10⁸ cells). In contrast, the sample amount generated by lab animals and patients is very limited. For example, a core needle biopsy generates a total of 10⁴-10⁵ cells. Circulating tumor cells are present by the frequency of 1-10 per ml of whole blood in patients with metastatic cancer. The sensitivity issue has hindered clinical research using patient materials and made patient stratification based on epigenomics impractical. Second, these assays typically require manual procedures for a duration of 3-4 days (not including library preparation and sequencing) and are not suitable for high-throughput data production. Rapid characterization of a large number of samples is vital for use of epigenomic knowledge in clinical research and patient stratification, because 1) epigenomic profiles vary among individual subjects, cell/tissue types and disease/developmental stages; 2) there are also tens to hundreds of histone marks of interests.

Microfluidics has been shown to provide a powerful platform for conducting low-input genomic, transcriptomic, proteomic and epigenomic analysis. There have been a few different attempts to utilize microfluidics to improve the ChIP process. Wu et al. developed an automated microfluidic device capable of performing ChIP-qPCR analysis using only 2000 cells, and was able to scale their platform to 16 simultaneous reactions (Wu, A. R. et al. Automated microfluidic chromatin immunoprecipitation from 2,000 cells. (Lab on a Chip 9, 1365, doi:10.1039/b819648f (2009); Wu, A. R. et al. High throughput automated chromatin immunoprecipitation as a platform for drug screening and antibody validation. Lab on a Chip 12, 2190-2198, doi:10.1039/c21c21290k (2012)). In work by inventors of the instant application, microfluidic ChIP-qPCR sensitivity was further reduced to 50 cells (Geng, T. et al. Histone modification analysis by chromatin immunoprecipitation from a low number of cells on a microfluidic platform. Lab Chip 11, 2842-2848, doi:10.1039/c11c20253g (2011)). Recently, combining a packed bed with a shear stress inducing oscillating washing step, it was demonstrated generating high-quality ChIP-seq data using as few as 100 cells (Cao, Z., Chen, C., He, B., Tan, K. & Lu, C. A microfluidic device for epigenomic profiling using 100 cells. Nat Methods 12, 959-962, doi:10.1038/nmeth.3488 (2015)).

Rotem et al., using a droplet microfluidic platform, was able to barcode chromatin from single cells and then perform ChIP-seq (Drop-ChIP)(Rotem, A. et al. Single-cell ChIP-seq reveals cell subpopulations defined by chromatin state. Nat Biotechnol 33, 1165-1172, doi:10.1038/nbt.3383 (2015)). While useful for understanding single cell heterogeneity, Drop-ChIP yielded a low number of reads per single cell (1000 unique reads per cell) and required a large pooling of single cells (on the order of 10³ cells) to generate datasets of sufficient quality as reference epigenomes. Other state-of-the-art ChIP technologies (iChIP, FARP-ChIP, LiNDA, nano-ChIP) all work at 500-10,000 cells per assay range. While high throughput analysis does exist in the form of AHT-ChIP, the analysis requires 100 million cells per assay, more than traditional ChIP-seq.

In previous works, an inventor of the instant invention designed a packed bed of immunomagnetic beads for highly efficiently collection of ChIP DNA that enabled ChIP-seq using as few as 100 cells. In spite of the high adsorption efficiency at the theoretical limit, a packed bed of beads creates substantial pressure drop in the microfluidic structure, and this problem would be further confounded were running multiple units in parallel attempted.

### SUMMARY

According to an aspect of some embodiments of the invention, the invention demonstrates Low Input Fluidized bed Enabled ChIP (LIFE-ChIP), a microfluidic platform for running multiple parallel ChIP assays simultaneously by utilizing microfluidic fluidized beds. Our device permitted running 4 ChIP-seq assays in one run with as few as 50 cells per assay. The supporting data has high reproducibility among various individual units and devices. An exemplary LIFE-ChIP assay could be finished in as little as 1 hour. The technology paves the way to high-throughput epigenomic profiling required by precision medicine.

There are several distinct advantages associated with LIFE-ChIP-seq. First, this fluidized-bed based ChIP technology permits running multiple parallel assays simultaneously. Such a feature is critical for high-throughput processing of samples needed for the examination of patient materials in the precision medicine settings. Furthermore, the fluidized bed technology effectively alleviates pressure building associated with bead manipulation in a microfluidic system.

Exemplary embodiments may permit production of multiple replicates from one sample and/or examination of multiple samples, all with a single device. A prototype embodiment detailed in the Examples demonstrates producing 4 replicates on one type of histone modification or 2 replicates each on two types of histone modifications from one sample.

LIFE-ChIP-seq is also a low-input technology. A prototype embodiment produced high-quality data with 100 or less cells per assay. This is comparable to or better than other state-of-the-art ChIP-seq technologies such as MOWChIP-seq and iChIP (Cao, Z., Chen, C., He, B., Tan, K. & Lu, C. A microfluidic device for epigenomic profiling using 100 cells. Nat Methods 12, 959-962, doi:10.1038/nmeth.3488 (2015); Lara-Astiaso, D. et al. Immunogenetics. Chromatin state dynamics during blood formation. Science 345, 943-949, doi:10.1126/science.1256271 (2014)). The low-input characteristic of exemplary embodiments is important for profiling primary cell types with low abundance.

An exemplary microfluidic process of LIFE-ChIP-seq may be largely automated. There is very little input from the operator during the process. Thus the technology may largely eliminate human errors and save labor.

Generally, a processing of using an exemplary microfluidic device comprises on-chip steps of loading of antibody-coated beads into a LIFE-ChIP-seq platform containing multiple parallel unit; flowing of the chromatin fragments through the fluidized beds (i.e. chromatin immunoprecipitation), and flowing of washing buffer through the fluidized beds for removing nonspecifically bound chromatin. Rotating schemes may be applied during loading and washing steps to keep even distribution of reagents among chambers and good fluidization of the beads.

An exemplary microfluidic device may comprise a plurality of chambers configured to accommodate fluidized beds for chromatin immunoprecipitation assays; one or more inlet ports configured or configurable to be in fluid communication with the plurality of chambers; a plurality of micromechanical valves actuatable in different combinations to control and change which of the plurality of chambers are in fluid communication with the one or more inlet ports and which of the plurality of chambers are not in fluid communication with the one or more inlet ports at different stages of operation; a magnetic field generating device for generating a magnetic field in the plurality of reaction chambers for manipulating magnetic beads; and a plurality of outlet ports for recovering contents from the chambers.

An exemplary method of performing chromatin immunoprecipitation (ChIP) for multiple parallel assays may comprise loading a plurality of parallel units with antibody-coated immunomagnetic beads; flowing chromatin molecules from an inlet into fluidized beds containing the immunomagnetic beads to form chromatin-bead conjugates; washing the chromatin-bead conjugates with a plurality of sequential wash buffers flowed through the fluidized beds; and collecting washed chromatin-bead conjugates for DNA release.

### BRIEF DESCRIPTION OF THE DRAWINGS

FIG. 1a. 2D top view schematic of an exemplary microfluidic LIFE-ChIP device. A glass slide that is bonded to the PDMS part is not shown.

FIG. 1b. Cross-sectional view of the LIFE-ChIP device showing the various layers of materials involved and the orientation of the magnet. Either the north or south pole of the magnet can be adjacent to the glass substrate of the microfluidic device.

FIG. 1c. 3D perspective view of the microfluidic networks within the LIFE-ChIP device with a two-layer structure. The valves 102 are all in the control layer and the rest of the structures are in the fluidic layer. A glass slide that is bonded to the PDMS part is not shown.

FIGS. 2a-2c. Overview of an exemplary LIFE-ChIP-seq device operation. Dark conduits are pressurized channels/structures in control layer, while transparency indicates unpressurized structures in control layer. The schematic omits details on the rotating schemes for loading and washing (see methods).

FIG. 3. is an exemplary microfluidic system.

FIG. 4a. Immunomagnetic bead fluidization with different magnet positions and total flow rates. Overview of magnet positions examined. Positions are denoted by the line corresponding to the bottom most edge of the magnet.

FIGS. 4b-4d. Fluidized beds generated with the magnet at positions 1-3 respectively. For each magnet position, beds were fluidized at increasing flow rates until significant bead loss was noted.

FIG. 5. Illustrates operation of a microfluidic device with two types of beads.

FIG. 6. A tapestation electropherogram of sheared chromatin prior to ChIP process. The average chromatin fragment size is 283 bp and was primarily between 150 and 600 bp.

FIGS. 7a-7c. Summary of H3K4me3 ChIP-seq data generated using LIFE-ChIP-seq platform with various sample sizes (50-1000 cells per assay). All samples were run using 120 μl loading volume, and 20 min per washing buffer. (a) Normalized LIFE-ChIP sequencing data at various cell numbers per assay compared with ENCODE data (ENCFF825QGB, ENCFF598WCX by Bradley Bernstein group) (The ENCODE project consortium. An integrated encyclopedia of DNA elements in the human genome. Nature 489, 57-74. doi:10.1038/nature11247 (2012)). (b) Pearson correlation matrix of ChIP-seq data for 1000, 300, 100, and 50 cell samples generated on single multiplexed chips compared with ENCODE data. Data generated by individual chambers/units are denoted as C1-C4. (c) Receiver Operating Curves for comparing LIFE-ChIP-seq to MOW-ChIP-seq data. MOWChIP-seq data and the ROC curve plotting method are from a previous publication by the inventor (Cao, Z., Chen, C., He, B., Tan, K. & Lu, C. A microfluidic device for epigenomic profiling using 100 cells. Nat Methods 12, 959-962, doi:10.1038/nmeth.3488 (2015)).

FIG. 8. Inter-device and inter-unit reproducibility of our data. All samples were run using the given cell number, 120 μl loading volume, and 20 min per washing buffer. Two LIFE-ChIP devices were operated at each of the following cell numbers per assay: 1000, 300, and 100.

FIGS. 9a and 9b. LIFE-ChIP-seq data with different loading volumes (30 and 120 μl). All samples were run using 1000 cells, the given loading volume, and 20 min per washing buffer. (a) Normalized LIFE-ChIP-seq tracks and (b) Pearson correlation matrix.

FIGS. 10a and 10b. LIFE-ChIP-seq data with washing conditions ranging from no washing to 30 min per washing buffer. All samples were run using 1000 cells and 120 μl loading volume. (a) Normalized LIFE-ChIP-seq tracks and (b) Pearson correlation matrix.

FIGS. 11a and 11b. LIFE-ChIP-seq data with different total assay times. All runs were taken using 300 cells per assay. 1 h assays were performed with 30 μl loading volume (i.e. 30 min for loading) and 10 min per wash buffer while 3 h assays performed with 120 μl loading volume (120 min for loading) and 20 min per wash buffer. a) Normalized LIFE-ChIP-seq tracks and (b) Pearson correlation matrix.

FIGS. 12a and 12b. LIFE-ChIP-seq for dual-bead loading and dual-histone-mark profiling. (a) Two-step process for loading two types of beads (targeting H3K4me3 and H3K27ac) into the system. (b) Genome browser tracks comparing our H3K4me3 and H3K27ac data to ENCODE data (ENCFF598WCX and ENCSR000AKC) at two loci in the genome. (The ENCODE project consortium. An integrated encyclopedia of DNA elements in the human genome. Nature 489, 57-74. doi:10.1038/nature11247 (2012)). (c) Pearson correlation matrix comparing H3K4me3 and H3K27ac data sets generated in one dual-bead LIFE-ChIP experiment.

### DETAILED DESCRIPTION

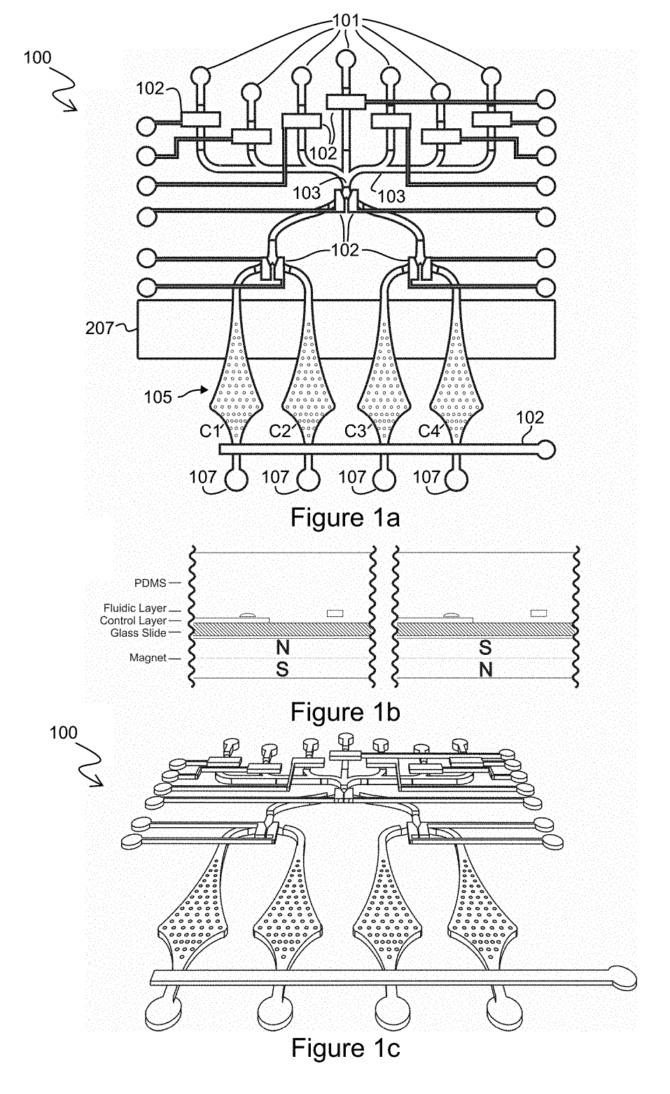

FIGS. 1a and 1b show a schematic of an exemplary microfluidic LIFE-ChIP device. The microfluidic device 100 is configured with a fluidized bed design with parallel units and common inlets. A fluidized bed generally refers to a suspension of solid particles in fluid contained in a reactor. Generally, an exemplary microfluidic device comprises a plurality of reaction chambers 105 (respectively C1, C2, C3, C4), one or more common inlet ports 101, a plurality of valves 102, and a plurality of outlet ports 107. As illustrated, the device 100 has 7 inlet ports 101 connected to 4 bell-shaped reaction chambers 105 through a series of splitting channels 103, and each reaction chamber 105 has a single outlet port 107. To avoid overcrowding the drawings, not every valve 102 or split 103 is individually labeled. However, seven valves 102 are provided with each controlling whether a respective inlet 101 is or is not in fluid communication with downstream elements (e.g., an adjacent split 103 or reactor 105). Another six valves 102 are provided as three pairs, each pair controlling the left and right branches of a split 103. A single valve 102 is provided which can simultaneously open or close the outlet ports 107. In total the device 100 contains a number of valves equal to the number of inlets+twice the number of divergent splits+at least one for the outlets. Altogether the device 100 as illustrated in FIGS. 1a and 1b contains 14 valves 102. Alternative embodiments (not illustrated) may have alternative valve configurations with fewer or greater valves irrespective of whether they also contain the same, fewer, or greater a number of reaction chambers or inlets. A permanent magnet 207 is placed adjacent to the reaction chambers 105 to generate magnetic field within the reaction chambers 105.

The microfluidic device 100 comprises two layers: a control layer which can be pressurized and a fluidic layer which allows the flow of reagents, molecules and particles. The fluidic layer comprises the inlet ports 101, channels 103, reaction chambers 105, and outlet ports 107. The control layer comprises the valves 102, which in some exemplary embodiments may be micromechanical valves. Together the two layers (fluidic layer and control layer) allow for fluid/particle manipulation within the channels 102 and reaction chambers 105 via the micromechanical valves 102. The control layer is adjacent and in a fixed position with respect to the fluidic layer. As non-limiting exemplary dimensions, structures of control layer may be, for example, 50 μm to 20 μm in depth. FIG. 1b is 3D schematic of the device 100 showing exemplary relative channel depths and geometric dimensions with the control layer on top of the fluidic layer. As detailed in a previous study (Unger, M. A., Chou, H.-P., Thorsen, T., Scherer, A. & Quake, S. R. Monolithic Microfabricated Valves and Pumps by Multilayer Soft Lithography. Science 288, 113-116, (2000)), the valves 102 are operated by exerting a pressure in the control layer that deforms the elastic material in between the control and fluidic layers and effectively collapses the section of fluidic channel adjacent to the valve 102 (i.e. to close the valve). The fluidic channel is open when such pressure is not exerted. It will be understood by those in the art that other types of micromechanical valves can be used in these cases without involving the two-layer structures (e.g. screw-based valves described in Hulme et al. Incorporation of prefabricated screw, pneumatic, and solenoid valves into microfluidic devices. Lab on a Chip 9, 79-86, (2009).).

As illustrated by FIGS. 1a and 1b, an exemplary individual chamber is bell-shaped to allow for maximum fluidization while avoiding bead loss. While other shapes may be employed in alternative embodiments, bell-shaped reaction chambers are advantageous. As exemplary dimensions, a prototype bell-shaped reaction chamber had a major axis of 9 mm and a minor axis of 3.5 mm. A range of 1:1 to 10:1 for major axis: minor axis ratio may be suitable in some embodiments. As evidenced by FIGS. 1 and 1b, the bell-shape is substantially two-dimensional, the height dimension generally being unchanged over the area of the bell. As a result the chamber assume a “bell” shape as viewed from above or below but not from the side. The floor and roof of a chamber may be flat (e.g., a flat plane). The reaction chambers may include microscale pillars (with a diameter of, e.g., 140 μm) to prevent collapse. The reaction chambers may have a depth of, for example, 50 μm, while the parts that formed fully closed valves may have a smaller depth of 25 μm, for example. The chamber and channel depths may be in the range of 5 to 200 μm. While the bell shape has been found to be advantageous, some embodiments may employ shape alternatives to the bell shape. A chamber 105 may itself be regarded as a type of channel.

A microfluidic device 100 is configured for performing an assay or part thereof, in particular a ChIP-seq assay or a part thereof. Generally, a ChIP-seq assay for examining histone modifications typically involves several steps: i) cross-linking to fix histones to DNA sequences that they interact with; ii) sonication or enzymatic digestion to generate chromatin fragments; iii) chromatin immunoprecipitation (ChIP) to adsorb chromatin fragments containing the histone of interest on immunomagnetic beads that are functionalized with an antibody targeting a specifically modified histone; iv) release of the DNA fragments (i.e. ChIP DNA) from bead surface; v) sequencing of the DNA fragments and establishing genome-wide profile for the histone modification. Exemplary microfluidic devices according to the invention may perform one more of these steps. In particular, an exemplary microfluidic device 100 may perform at least step (iii), namely chromatin immunoprecipitation. Other steps (e.g., (i), (ii), (iv), and/or (v)) may be perform off-chip or on-chip. Variations of the microfluidic device 100 depicted in the figures may be made within the spirit and scope of the invention to include one or more of the remaining ChIP-seq steps on-chip or off-chip with respect to the chromatin immunoprecipitation step.

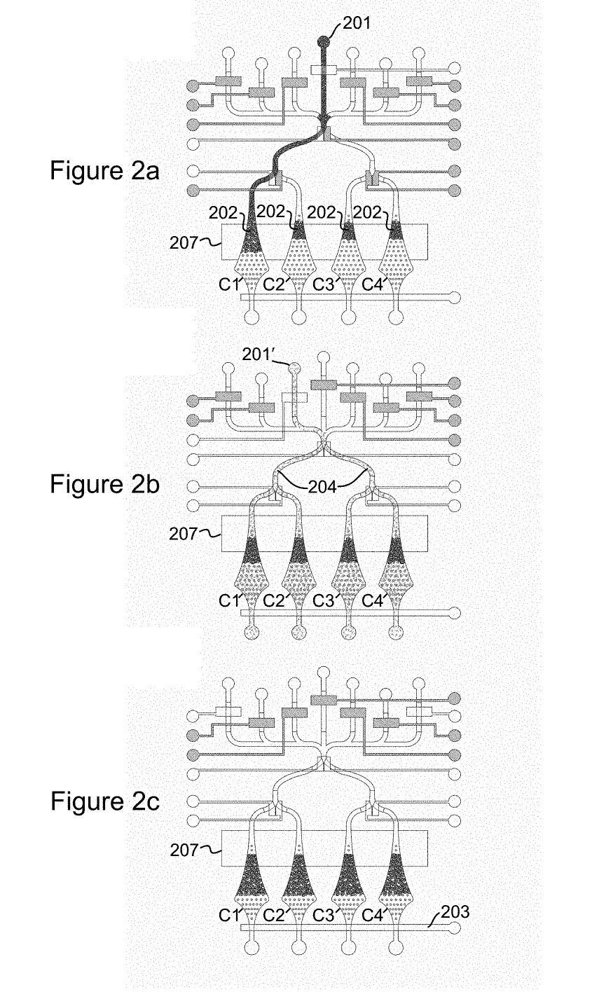

Exemplary operation of a microfluidic device 100 is demonstrated in FIGS. 2a to 2c. The platform (that is, the device) may be operated in several steps to perform chromatin immunoprecipitation (ChIP). Generally, FIG. 2a depicts loading of antibody-coated magnetic beads into a LIFE-ChIP-seq platform containing 4 parallel units; FIG. 2b depicts flowing of the chromatin fragments through the fluidized beds (i.e. chromatin immunoprecipitation); and FIG. 2c depicts flowing of washing buffer through the fluidized beds for removing nonspecifically bound chromatin. The process will now be explained in greater detail.

First, as shown in FIG. 2a, magnetic beads coated with antibody are loaded into the respective units (4 units in this example). In this context a “unit” comprises or consists of a chamber, in particular a reaction chamber. The units may be loaded from a single common inlet port 201. The loading may be performed with a constant flow rate (e.g., 8 μl/min). A rotating scheme may be used for loading the units C1, C2, C3, and C4. For instance, the bead suspension 202 may be flowed into an inlet port at a constant flow rate while the valves alternate the flow path among the chambers at intervals to ensure even loading. Exemplary beads usable in embodiments are magnetic beads coated with antibody. The beads may be superparamagnetic.

The beads may be referred to as immunomagnetic beads, or magnetic immunoprecipitation beads. For the sake of uniformity, most of this description uses the term “immunomagnetic beads,” but different types of beads may be employed in different embodiments.

To use specific conditions as a non-limiting example, a bead suspension (6 μg/μl) may be flowed into one of 4 reaction chambers at a flow rate of 8 μl/min while the valves alternated flow among the chambers in the order of chamber C1-C3-C2-C4 at 4 s intervals to ensure even loading. In a prototype embodiment using the conditions just identified, each loading cycle took 16 s (4 sec for each of the 4 chambers) and all 4 chambers were finished loading in 20 cycles. During the loading process a magnet 207 is situated adjacent to the reaction chambers. The magnet may be repositionable with respect to the plurality of reaction chambers. As depicted in FIG. 2a, the magnet 207 may be positioned near a top side of the chambers (“top” in this context being used to describe an end of a reaction chamber nearest to the inlet ports, which illustratively is also the end of a reaction chamber nearest the top of the paper on which the figures appear). The magnet 207 is for manipulating the immunomagnetic beads within the plurality of reaction chambers. The force field generated by magnet 207 and the drag force generated by the fluid flow help control the density of the immunomagnetic beads, in particular their packing. The magnet 207 may be any suitable magnetic field generating device. Non-limiting examples include a permanent magnet and an inductor coil. In any case the magnet 207 is configured for generating a magnetic field in the plurality of reaction chambers for manipulating the magnetic beads.

Next, as depicted by FIG. 2b, crosslinked and sheared chromatin 204 (e.g., 150-600 bp) is flowed into a common inlet 201′. To ensure even loading of chromatin among chambers and proper fluidization, a constant flow rate may be delivered to the inlet 201′ and valve actuation cycled to produce a rotating loading scheme. An exemplary flow rate for this step is 1 μmin. In some instances all four reaction chambers may be loaded simultaneously, e.g., after a period of rotating loading. FIG. 2b depicts a simultaneous loading condition. As apparent from a comparison of FIGS. 2a and 2b, the magnet 207 may be moved to a different position for the chromatin loading. For example, the magnetic 207 may be moved to a position further from the top of the reaction chamber. Despite the fact the beads are retained by the magnet in a specific subregion of the available space of a reaction chamber, the beads are preferably kept as a fluidized bed and therefore not “packed” in an extreme sense of the word.

The chromatin loading step allows targeted chromatin fragments to adsorb on the beads' surfaces (i.e. ChIP). Sufficient time is allowed for immunoprecipitation, e.g., 30 to 120 minutes. Broadly speaking, immunoprecipitation time may be 10-180 minutes. A narrower exemplary range is 30-60 minutes. For example, immunoprecipitation time may be no longer than 10, 15, 20, 25, 30, 35, 40, 45, 50, 55, or 60 minutes from start to completion. An exemplary time breakdown for a microfluidic device with 4 reaction chambers is as follows. Chromatin solution is directed into one of 4 reaction chambers while the on-chip valves alternated flow among the chambers in the order of chamber C1-C3-C2-C4 with intervals varying from 8 to 0.25 s for a total of 104 s (cycle 1: 8 s per chamber, cycle 2: 6 s per chamber, cycle 3: 4 s per chamber, cycle 4: 3 s per chamber, cycle 5: 2 s per chamber, cycle 6: 1 s per chamber, cycle 7-8: 0.5 s per chamber, cycle 9-12: 0.25 s per chamber). After these 12 loading cycles (i.e. rotating scheme 2), all chambers are opened to load chromatin into them simultaneously (FIG. 2b). This rotating loading scheme 2 was performed every 15 min of loading to reduce bead settling.

After the chromatin loading and adsorption, wash buffers are flowed sequentially through the fluidized beds of the reaction chambers, as depicted in FIG. 2c. Individual inlet ports may be used for respective wash buffers, or combinations of inlet ports may be used. In some exemplary embodiments at least three different wash buffers are administered: an IP buffer, a low salt buffer, and a high salt buffer. As was the case with the chromatin loading step, washing may be performed using a rotating delivery scheme, simultaneous delivery scheme, or a combination thereof. For example, a non-limiting example embodiment did the following. For each respective wash buffer, a rotating scheme was applied at the start of the washing period. Washing buffer was directed into one of 4 reaction chambers while the on-chip valves alternated flow among the chambers in the order of chamber C1-C3-C2-C4 with intervals varying from 4 to 0.25 s for a total of 50 s (cycle 1: 4 s per chamber, cycle 2: 3 s per chamber, cycle 3: 2 s per chamber, cycle 4: 1.5 s per chamber, cycle 5: 1 s per chamber, cycle 6: 0.5 s per chamber, cycle 7-8: 0.25 s per chamber). The rotating scheme also included flow rate change within the first 50 s. The flow rate was slowly ramped from 1 to 5 μl/min during the period, increasing by 1 μl/min per 10 s. All chambers were then open after the first 50 s, and the flow rate was ramped from 5 to 20 μmin during the next 50 s (i.e. increasing by 3 μl/min per 10 s). For the rest of the washing period, a constant flow rate of 20 μmin was used with all the chambers open. A rotating scheme was applied again in the first 100 s every time the device was switched to a new wash buffer. All told, 3 wash buffers (IP, low salt, and high salt) were flowed sequentially through the fluidized beds from different inlets, at a flow rate of 20 μl/min for 10-30 min each.

After performing all washing steps, the beads may be collected from the device outlet ports 107. This may be accomplished by removing the magnet 207, opening the valve 203 (if not already open) configured for closing off the reaction chambers from the outlet ports, and flowing an IP buffer to flush the beads from the reaction chambers. Exemplary conditions for this collection step are, for example, a flushing rate of 400 μl/min for 15 s.

The washed and collected chromatin-bead conjugates may then be subject to ChIP DNA release (off-chip) and sequencing library preparation. These steps may be performed according to known best practices. One example is detailed below in the Examples section.

A microfluidic device according to the invention may comprise a controller or be an element of a system which further includes a controller. The controller is attached or attachable to the control layer of the microfluidic device to control the valves and may also control the liquid delivery into the inlets and liquid removal from the outlets.

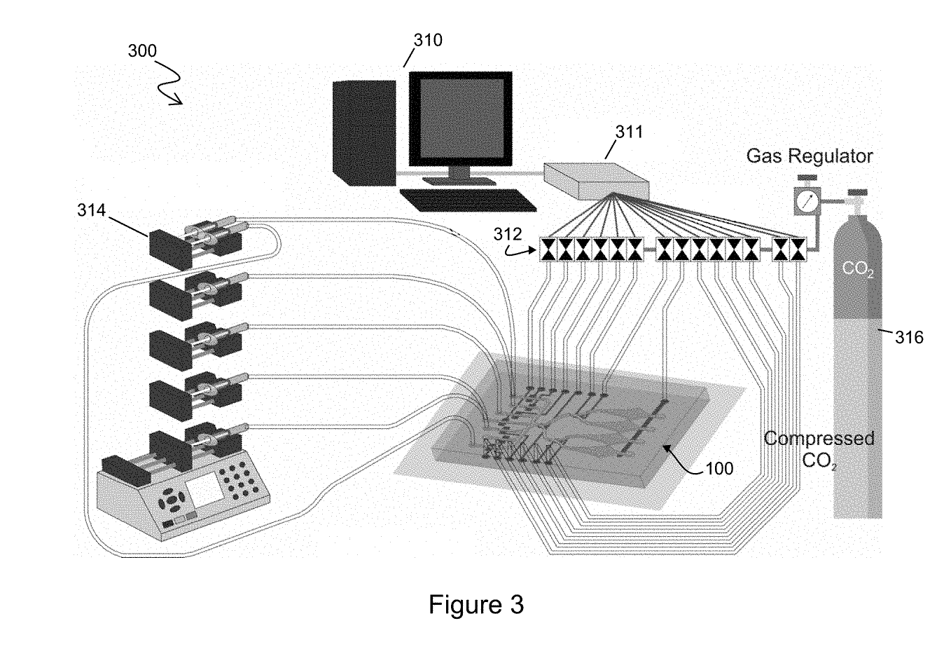

FIG. 3 shows an exemplary microfluidic system 300. The microfluidic system 300 shown in FIG. 3 comprises a microfluidic device 100 (i.e., microfluidic chip), a controller 310 (e.g., a computer running a custom LabVIEW program and supporting components like a data acquisition card 311), a set of solenoid valves 312 (e.g., ASCO) controlled by the controller 310, syringe pumps 314 (e.g., Chemyx) controlled by the controller 310, and a pressure source 316 (e.g., gas cylinder). While specific numbers of each element are illustrated in FIG. 3, it will be understood by those in the art that fewer or greater numbers of respective elements may be used in the practice of the invention. For example, a controller 310 may consist of or comprise multiple control elements which may or may not be interconnected. In addition, respective components may be substituted or eliminated by other devices suitable to the described functionality. For example, a controller 310 may be a standalone computer, a special purpose computer or microcontroller, one or more processors, a network, or a combination thereof. Syringe pumps 314 may be any suitable microfluidic flow control apparatus capable of delivering controlled flows such as specific flow rates identified elsewhere in this disclosure by way of example. Solenoid valves 312 and pressure source 316 may be any suitable actuating apparatus capable of actuating the on-chip micromechanical valves 102 (see FIGS. 1a and 1b). The microfluidic device 100, in particular the control layer thereof, may be configured to operate using a constant pressure (e.g., 30 psi) supplied by the gas source. Elements of system 300 are connected with tubing (e.g., PFA tubing) suited for the solutions and/or pressures being delivered to microfluidic device 100. If desired a visual monitoring system may be arranged with optical components including, for example, microscopic lenses (not shown in FIG. 3).

Exemplary systems, devices, and methods may be characterized according to a number of parameters, including input volume flow rate (of respective reagents, solutions, suspension, buffers, etc. admitted to the fluidic layer through one or more inlet ports), magnet location or locations (at one or more stages of the process), washing time, and overall time of the LIFE-ChIP assay, among others. Exemplary operational conditions encourage high levels of fluidization for washing and a dense packing of beads for the chromatin loading step, all while avoiding bead loss in each chamber.

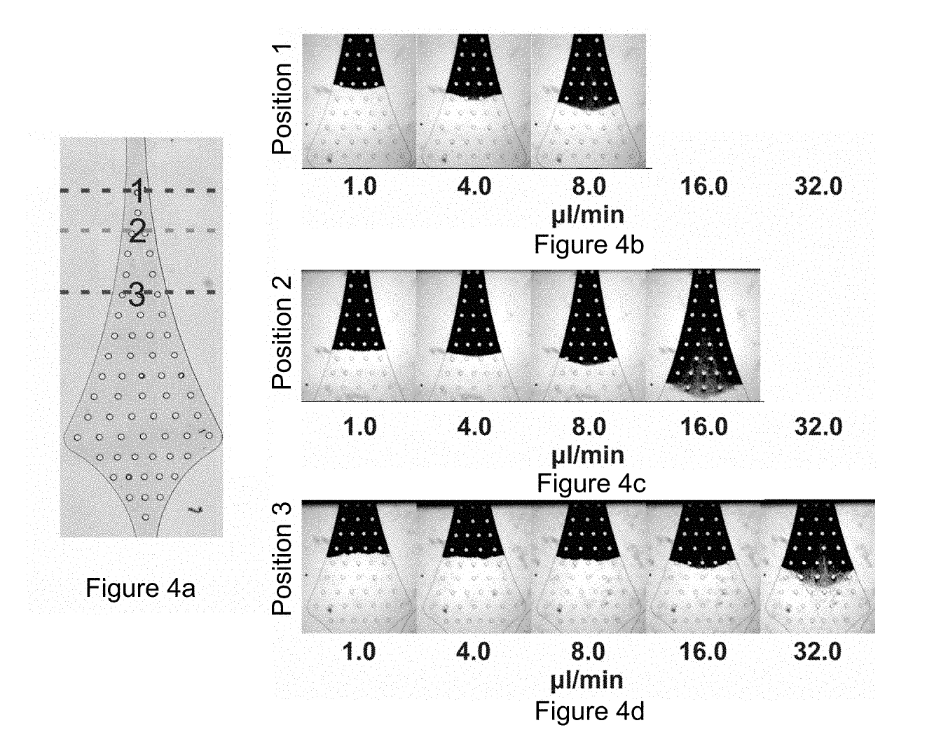

Operational conditions including flow rate and magnetic field location are summarized in FIGS. 4a to 4d. FIGS. 4a to 4d are photographs of a prototype reaction chamber with immunomagnetic bead fluidization with different magnet positions and total flow rates. FIG. 4a provides an overview of magnet positions examined during prototype implementation. Positions are denoted by the line corresponding to the bottom most edge of an external magnet. FIGS. 4b to 4d show fluidized beds generated with the magnet at positions 1-3, respectively. For each magnet position, beds were fluidized at increasing flow rates until significant bead loss was noted.

Of the three magnet positions identified in FIG. 4a, magnet position 3 was selected as a preferred configuration as it allowed for a fluidized bed with high bead density for chromatin loading and a highly fluidized bed for washing while having a large range of fluidization flow rates without bead loss. We used an inlet flow rate of 1 μl/min (i.e. 0.25 μmin through each of the 4 units) for chromatin loading (with a possible range of 0.5-5 μl/min) and 20 μl/min (with a possible range of 10-100 μl/min) for washing. The 20 μl/min flow rate for washing created beds that retained most beads with very slight variance in the flow rate among units which was caused by slight variability in the volume of beads in each chamber.

Washing time may be characterized as the duration for flowing each wash buffer through fluidized bed to remove nonspecifically adsorbed chromatin fragments. Washing removes nonspecifically adsorbed chromatin and improves enrichment of ChIP DNA. However, excessive washing can decrease the amount of ChIP DNA collected thus require additional amplification and result in lower-quality sequencing library. Washing times may be for, example, between 0 and 30 min with 1000 cells per assay (unit), more preferably 10 to 30 min. No washing, while possible, is generally disadvantageous because it generally yields lower-quality data. By contrast washing times from 10 to 30 min generally yield high-quality data. Similarities between data obtained with washing times 10-30 min suggested that the fluidized bed washing was effective for removing nonspecific chromatin molecules (see EXAMPLES below). A fluidized bed implemented in LIFE-ChIP decreases the possibility for physical trapping as compared to prior packed bed reactors.

Depending on the configuration of specific parameters discussed above for individual alternative embodiments, the total time of a LIFE-ChIP assay may vary. However, exemplary total on-chip times range from 1 hour to 3 hours, for example, with acceptable quality of resulting ChIP-seq data. The 1 to 3 hr duration includes multiple parallel assays, e.g., two, four, or more than four. An exemplary microfluidic device is scalable to tens (e.g., at least 10, 20, 30, 40, 50, 60, 70, 80, 90), hundreds (e.g., at least 100, 200, 300, 400, 500, 600, 700, 800, 900), or more parallel assays. Generally speaking, an exemplary microfluidic device is scalable to any size.

An exemplary method of manufacturing a fluidic device like device 100 shown in the figures is by multi-layer soft lithography. The fluidic layer, the control layer, or both layers may be monolithic, individually or collectively. Suitable materials include, for example, polymers (e.g., polydimethylsiloxane, PDMS). A specific example manufacturing method employed with a test prototype is described below in the EXAMPLES.

Different types of beads may be used with embodiments of the invention. In particular, two or more (e.g., 2, 3, 4, 5, 10, 20, 50, or more) types of beads with different antibody-coatings may be used in a single run on a single microfluidic device 100.

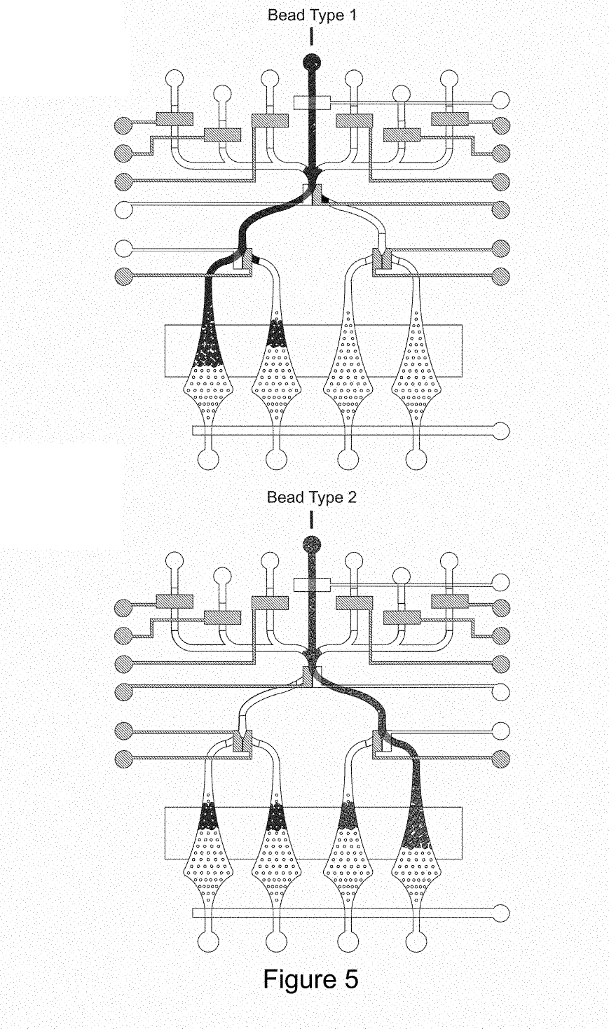

FIG. 5 shows two types of antibody-coated beads (for different respective histone modifications of interest) loaded from two separate inlets sequentially while avoiding cross contamination. A parallel ChIP-seq run is performable to generate data sets for separate histones of interest, each with multiple replicates (e.g., two replicates per the illustrated example), using low input quantities (e.g., 300 or fewer cells per assay, 100 or fewer cells per assay).

### EXAMPLES

### Example 1. Prototype Microfluidic Device, System, and Use

Following is a non-limiting example demonstrating manufacture, use, and performance of a prototype microfluidic device consistent with the foregoing descriptions and in particular the figures. The example is demonstrative that an exemplary microfluidic device performs well with low-input (e.g., <100 cells per assay) and high-throughput (e.g., four assay in an hour). LIFE-ChIP-seq data are deposited under accession number GSE 102932 at Gene Expression Omnibus (https://www.ncbi.nlm.nih.gov/geo/query/acc.cgi?acc=GSE102932).

### Device Fabrication

The microfluidic system consisted of 4 reaction chambers (units) connected to 7 input channels with successive splits. There were two-layered micromechanical valves (Unger, M. A., Chou, H.-P., Thorsen, T., Scherer, A. & Quake, S. R. Monolithic Microfabricated Valves and Pumps by Multilayer Soft Lithography. Science 288, 113-116, (2000).) to close/open each and every inlet and split. The bell-shaped reaction chambers had a major axis of 9 mm and a minor axis of 3.5 mm and included microscale pillars (with a diameter of 140 μm) to prevent collapse. The reaction chambers had a depth of 50 um, while the parts that formed fully closed valves had a depth of 25 μm.

The chip was manufactured using two-layer soft lithography (Unger, M. A., Chou, H.-P., Thorsen, T., Scherer, A. & Quake, S. R. Monolithic Microfabricated Valves and Pumps by Multilayer Soft Lithography. Science 288, 113-116, (2000).) in polydimethylsiloxane (PDMS, RTV615, Momentive Advanced Materials). Photomasks were prepared using LayoutEditor and printed on Mylar transparencies at high resolution (10,160 DPI) by FineLine Imaging. The photomasks were used to fabricate 2 separate master molds using photolithography on silicon wafers (3 inch, university wafer). The control layer and a part of the fluidic layer (some channels and reaction chambers) were fabricated using SU-8 2025 (MicroChem) to create rectangular channels (depth ˜50 um). The fully closed valves of the fluidic layer were fabricated by spinning 2 layers of AZ 9260 (EMD Performance Materials) each at 1500 RPM for 30 seconds, with a 10 min wait between the spins to allow evaporation of solvent (depth ˜20 um).

The fluidic layer master was then heated to 130° C. for 60 s to round the AZ channels. Prepolymer PDMS was mixed in a ratio of 5:1 prepolymer to crosslinking agent, degassed, and cast as a bulk layer (˜5 mm thick) on the fluidic layer mold, while a degassed 20:1 ratio of PDMS was spun onto the control layer mold at 1750 RPM for 30 s. After partial curing at 75° C. for 12 min, the fluidic layer casting was removed from the silicon master and inlet and outlet ports were punched using a 1.5 mm core punch (Harris Uni-Core). The fluidic layer was laid on top of the control layer (that was still attached to its master), aligned and cured at 75° C. for 1 h. The multilayer device was then removed from the mold and punched, using a 1.5 mm core punch. The PDMS device and a glass slide (50 mm×75 mm, Corning), precleaned with dish soap, were treated by plasma (Harrick Plasma) then bonded together irreversibly. The glass bound PDMS chips were cured at 75° C. for 30 min to strengthen bonding and reduce air bubbles before being used.

### Cell Culture

GM 12878 cells were obtained and cultured as described in Cao, Z., Chen, C., He, B., Tan, K. & Lu, C. A microfluidic device for epigenomic profiling using 100 cells. Nat Methods 12, 959-962, doi:10.1038/nmeth.3488 (2015). GM cells were cultured in RPMI 1640 supplemented with 15% FBS, 100 U penicillin, and 100 mg/mL streptomycin at 37° C. in a humid incubator with 5% CO₂. The cells were passaged every 2 d in order to maintain log phase growth.

### Chromatin Shearing

Sonicated chromatin was prepared as described in Cao, Z., Chen, C., He, B., Tan, K. & Lu, C. A microfluidic device for epigenomic profiling using 100 cells. Nat Methods 12, 959-962, doi:10.1038/nmeth.3488 (2015). Sample containing 10⁶ cells was used as the starting material and centrifuged at 1,600 g for 5 min and washed twice with 1 ml cold PBS. Cells were crosslinked by adding 1 ml freshly made 1% formaldehyde and incubated at room temperature for 5 min on a shaker. Crosslinking was promptly terminated by adding 50 μl of 2.5 M glycine and shaking for 5 min. The pellet was resuspended in 130 μl Covaris buffer (10 mM Tris-HCl, pH 8.1, 1 mM EDTA, 0.1% SDS and 1× protease inhibitor cocktail) and sonicated using the following conditions. Using Covaris M 220 (Covaris), the sample was sheared at 4° C. with 75 W peak incident power, 5% duty factor, and 200 cycles per burst for 12 min. The sheared sample was centrifuged in a 4° C. centrifuge at 14,000 g for 10 min, and the supernatant was transferred to a new tube. Sheared chromatin was diluted with IP buffer (20 mM Tris-HCl, pH 8.0, 140 mM NaCl, 1 mM EDTA, 0.5 mM EGTA, 0.1% (w/v) sodium deoxycholate, 0.1% SDS, 1% (v/v) Triton X-100, with 1% freshly added PMSF and PIC) and aliquoted into 25 μl samples containing 250, 500, 1500, and 5,000 cell samples and stored at

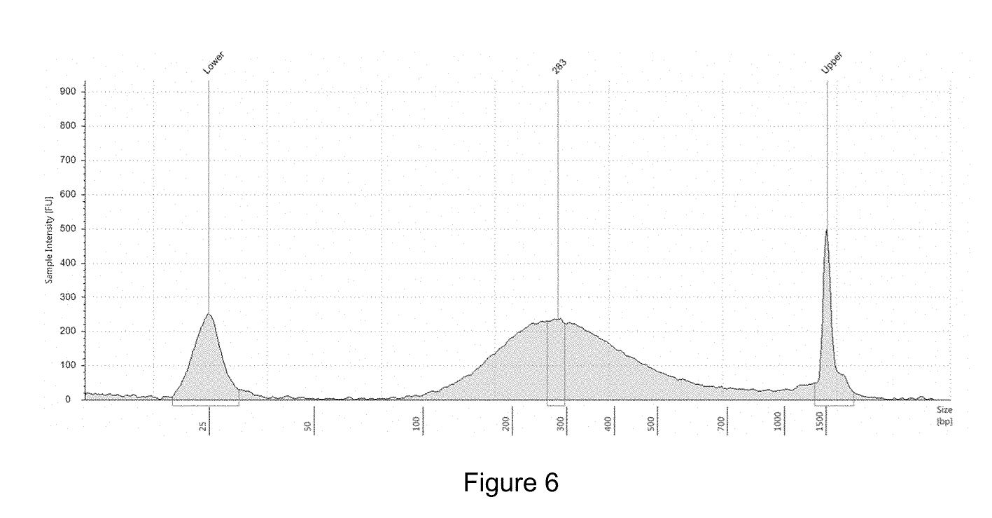

−80° C. Selective chromatin samples were purified by ethanol precipitation for fragment analysis using HS D1000 tapes on Tapestation 2200 (Agilent) (FIG. 6). Before use, 20% was taken out as an input sample and the remaining sample was diluted to the appropriate volume according to experimental procedure (30-120 μl).

### Bead Preparation

Protein A coated superparamagnetic Dynabeads (Invitrogen) were utilized for immunoprecipitation of sheared chromatin. 10 μl of bead suspension was washed twice with 100 μl freshly prepared blocking buffer (made by mixing 467 μl filtered PBS and 33 μl of 7.5% Bovine Albumin Fraction V solution) in each wash. The beads were incubated for 2 h on a rotator at 4° C. in 300 μl blocking buffer containing 5, 10, or 20 μl of H3K4me3 antibody (07-473, Millipore) for 50, 100-300, and 1000 cells per CUP assay, respectively. The beads were then washed twice in 100 μl IP buffer, before being resuspended in 50 μl IP buffer for on-chip use.

### Device Operation

A microfluidic system corresponding to system 300 in FIG. 3 was assembled. The prototype system comprised a microfluidic chip, a computer running a custom LabVIEW program, which controlled a set of 14 solenoid valves (ASCO) and 3 syringe pumps (Chemyx), and a gas cylinder which provided a pressure source. The gas pressure used throughout this experiment was 30 psi. The microfluidic chip was placed on an Olympus microscope IX71 with a CoolSNAP HQ2 camera (Photometrics) for visual monitoring of experiments. The solenoid valve system was connected to the microfluidic device with PFA high purity tubing (IDEX). Prior to the experiment, the control layer channels were filled with water. Washing buffers were loaded into syringes, while small-volume solutions (<200 μl) were loaded into the tubing connected to water filled syringes with an air gap to separate solution from water. The syringes were loaded onto the computer-controlled syringe pumps and connected to the microfluidic device through PFA tubing. The fluidic layer was evacuated of air by flowing IP buffer (20 mM Tris-HCl, pH 8.0, 140 mM NaCl, 1 mM EDTA, 0.1% Sodium deoxycheolate, 0.1% SDS, 1% (v/v) Triton X-100, with 1% freshly added PMSF and PIC) through the channels at a flow rate of 10 μl/min. After all air bubbles were evacuated from the fluidic layer, a ½×⅛×3 inch magnet (K&J Magnetics) was positioned under the reaction chambers with upper edge of the magnet aligned with position 2 (FIG. 4a, 2a). Operation was performed as demonstrated in FIG. 2. The beads were loaded into 4 chambers using “rotating scheme 1”. The bead suspension (6 μg/μl) was flowed into one of 4 reaction chambers at a flow rate of 8 μl/min while the valves alternated flow among the chambers in the order of chamber C1-C3-C2-C4 at 4 s intervals to ensure even loading. Each loading cycle took 16 s and loading all 4 chambers was finished in 20 cycles (FIG. 2a). Beads were visually inspected to ensure even loading in each chamber. The magnet was repositioned, with the lower edge aligning with position 3 in FIG. 4a. The chromatin was loaded at a flow rate of 1 μl/min. To ensure even loading of chromatin among chambers and proper fluidization, chromatin was initially loaded using “rotating scheme 2”. Chromatin solution was directed into one of 4 reaction chambers while the on-chip valves alternated flow among the chambers in the order of chamber C1-C3-C2-C4 with intervals varying from 8 to 0.25 s for a total of 104 s (cycle 1: 8 s per chamber, cycle 2: 6 s per chamber, cycle 3: 4 s per chamber, cycle 4: 3 s per chamber, cycle 5: 2 s per chamber, cycle 6: 1 s per chamber, cycle 7-8: 0.5 s per chamber, cycle 9-12: 0.25 s per chamber). After these 12 loading cycles (i.e. rotating scheme 2), all chambers were opened to load chromatin into them simultaneously (FIG. 2b). This rotating loading scheme 2 was performed every 15 min of loading to reduce bead settling. Washing was performed with IP buffer, low salt buffer (20 mM Tris-HCl, pH 8.0, 150 mM NaCl, 2 mM EDTA, 0.1% SDS, 1% (v/v) Triton X-100, with 1% freshly added PMSF and PIC), and high salt buffer (20 mM Tris-HCl, pH 8.0, 500 mM NaCl, 2 mM EDTA, 0.1% SDS, 1% (v/v) Triton X-100, with 1% freshly added PMSF and PIC) in that order (FIG. 2c). For each washing step, “rotating scheme 3” was applied at the start of each washing period. Washing buffer was directed into one of 4 reaction chambers while the on-chip valves alternated flow among the chambers in the order of chamber 1-3-2-4 with intervals varying from 4 to 0.25 s for a total of 50 s (cycle 1: 4 s per chamber, cycle 2: 3 s per chamber, cycle 3: 2 s per chamber, cycle 4: 1.5 s per chamber, cycle 5: 1 s per chamber, cycle 6: 0.5 s per chamber, cycle 7-8: 0.25 s per chamber). Rotating scheme 3 also included flow rate change within the first 50 s. The flow rate was slowly ramped from 1 to 5 μl/min during the period, increasing by 1 μl/min per 10 s. All chambers were then open after the first 50 s, and the flow rate was ramped from 5 to 20 μl/min during the next 50 s (i.e. increasing by 3 μl/min per 10 s). For the rest of the washing period, a constant flow rate of 20 μl/min was used with all the chambers open. Rotating scheme 3 was applied again in the first 100 s every time when a switch was made to a new wash buffer. After performing all washing steps, the beads were collected from the system by removing the magnet and flowing IP buffer at a rate of 400 μmin for 15 s. The beads were collected via pipetting at each chamber outlet.

### DNA Purification

CUP and input chromatin samples (beads or 10 μl solution, respectively) were incubated overnight at 65° C. with 200 μl (190 μl for input) elution buffer (200 mM NaCl, 50 mM Tris-HCl, 10 mM EDTA, 1% SDS, 0.1M NaHCO₃) supplemented with 2 μl of 10 μg/μl proteinase K. After incubation, DNA was isolated from protein debris and beads by adding 200 μl of Phenol:Chloroform:Isoamyl Alcohol 25:24:1 to the elution mix and vortexing. After a 5 min centrifuge at 16,100 g, the supernatant (200 μl) was removed and mixed with 750 μl 100% ethanol, 50 μl of 10 M ammonium acetate, and 2 μl of 5 μg/μl glycogen. The solution was vortexed and incubated at −80° C. for 2 h. After incubation, the samples were centrifuged at 16,100 g at 4° C. for 5 min. The supernatant was removed, and 500 μl of 70% ethanol was added to the tube without disturbing the pellet. After a 5 min centrifuge under the same conditions, the solution was removed, and the pellet air dried. The pellet was then dissolved into 40 μl of water for library preparation.

### Library Preparation

Library preparation was performed using the Swift Bio S2 library preparation kit (Swift Biosciences) using 40 μl of purified DNA. The manufacturer's instructions were followed with minor modifications. Specifically, 2.5 μl of 20× EvaGreen were added into the 50 μl amplification reaction mix, and amplification was terminated after samples saw a >3000 RFU increase (in a BioRad CFX Connect). After DNA purification with SPRI beads, DNA was then eluted into 7 μl low EDTA TE buffer where 2 μl could be used for qPCR analysis for preliminary quality control analysis, Kappa DNA quantification, and Tapestation fragment size analysis and the other 5 μl for library pooling. Libraries were pooled at 10 nM for sequencing by Illumina HiSeq 4000 with single-end 50 nt read.

### Read Mapping and Normalization

Sequencing reads were first trimmed using Trim Galore before being aligned to the hg19 genome using bowtie v2.4 with default settings. Normalized reads were computed across the genome in 25 bp bins according to the following equation:

$$Normalized Signal = {( \frac{Reads in bin}{Total uniquly mapped reads} \times 1000000 )}_{IP} - {( \frac{Reads in bin}{Total uniquely mapped reads} \times 1000000 )}_{Input}$$

For visualization, the reads were elongated to 250 bp reads to more accurately describe the read DNA before normalization and were normalized according to an input sample also with elongated reads. The normalized samples were converted to bigwig format using UCSC bedGraphtoBigWig.

### Peak Calling

Uniquely mapped un-normalized reads were used for peak calling. Peak calling was performed using MACS using a P value <10⁻⁵ with default settings.

### Determination of Pearson Correlation Coefficients

Correlation analysis was carried out in order to examine the consistency among different datasets. Promoter regions were defined as 2 kb upstream and downstream from the transcription start sites extracted from RefSeq data. The average normalized signal over each promoter region was quantified and the correlation was calculated using a custom Perl script.

### Results

The prototype LIFE-ChIP-seq platform consisted of 7 inlet ports connected to 4 bell-shaped reaction chambers through a series of splits (as depicted in FIGS. 1a and 1b). The individual chamber shape was optimized to allow for maximum fluidization while avoiding bead loss. The microfluidic device contained two layers (fluidic layer and control layer) for fluid/particle manipulation via micromechanical valves.

The operational conditions (flow rate and magnet location) summarized in FIGS. 4a to 4d were optimized for high levels of fluidization for washing and a tight packing of beads for the chromatin loading step, all while avoiding bead loss in each chamber. Magnet position 3 was used as it allowed for a tightly packed bed for chromatin loading and a highly fluidized bed for washing while having a large range of fluidization flow rates without bead loss. Inlet flow rate was 1 μl/min (i.e. 0.25 μl/min through each of the 4 units) for chromatin loading and 20 μl/min for washing. The 20 μl/min flow rate for washing created beds that retained most beads with very slight variance in the flow rate among units which was caused by slight variability in the volume of beads in each chamber.

The platform was operated in several steps (see FIGS. 2a to 2c). First, with a permanent magnet situated with the upper edge aligned with position 2 (see FIGS. 4a and 2a for reference), antibody-coated immunomagnetic beads were loaded into the 4 units from inlet 4 at a flow rate of 8 μmin while switching flow among units in the order of C1-C3-C2-C4 (FIG. 2a). Each loading session into one specific unit lasted 4 s and 20 loading cycles (16 s each) were used to load the chambers. Second, crosslinked and sheared chromatin (150-600 bp) was flowed from inlet 3 at 1 μl/min into the fluidized beds in the 4 units simultaneously (FIG. 2b). This step lasted 30 or 120 min to allow targeted chromatin fragments adsorb on the bead surface (i.e. ChIP). Finally, 3 wash buffers (IP, low salt, and high salt) were flowed sequentially through the fluidized beds from different inlets, at a flow rate of 20 μmin for 10-30 min each (FIG. 2c). The washed chromatin-bead conjugates were collected for ChIP DNA release (off-chip) and sequencing library preparation. Several rotating schemes were created and applied during loading and washing steps to keep even distribution of reagents among chambers and good fluidization of the beads.

Using the above-described platform, examination was made of tri-methylation of lysine 4 on histone H3 (H3K4me3) in GM12878 cells (a human B-Lymphocyte cell line). All data are summarized in Table 1.

TABLE 1
Summary of LIFE-ChIP-seq data. “1K_120L_0W_C1” depicts
LIEE-ChIP-seq done with 1K cell sample, 120 μl loading volume,
0 min per wash using chamber 1 of a device. Device number is
added when data taken under the same conditions using different
(that is, separate but identical) devices are included.

| | Aligned | Number | Redundancy |
|---|---|---|---|
| Sample | Reads (M) | Peaks | Rate |
| 1K_120L_0W_C1 | 7.6 | 17,166 | 13% |
| 1K_120L_0W_C2 | 8.1 | 28,954 | 12% |
| 1K_120L_0W_C3 | 12 | 19,397 | 13% |
| 1K_120L_0W_C4 | 7.6 | 18,118 | 10% |
| 1K_120L_10W_C1 | 23.1 | 39,416 | 27% |
| 1K_120L_10W_C2 | 12.9 | 40,885 | 18% |
| 1K_120L_10W_C3 | 20.8 | 45,921 | 20% |
| 1K_120L_10W_C4 | 14.9 | 41,646 | 21% |
| 1K_30L_20W_C1 | 24.5 | 21,162 | 19% |
| 1K_30L_20W_C2 | 29.9 | 23,810 | 14% |
| 1K_30L_20W_C3 | 32 | 37,687 | 32% |
| 1K_30L_20W_C4 | 25.4 | 37,176 | 24% |
| 1K_120L_20W_C1_Device1 | 15.4 | 45,691 | 26% |
| 1K_120L_20W_C2_Device1 | 20.8 | 23,143 | 43% |
| 1K_120L_20W_C3_Device1 | 8.4 | 22,906 | 24% |
| 1K_120L_20W_C4_Device1 | 10 | 29,172 | 24% |
| 1K_120L_20W_C1_Device2 | 18.5 | 44,978 | 23% |
| 1K_120L_20W_C2_Device2 | 16.6 | 23,987 | 22% |
| 1K_120L_20W_C3_Device2 | 19 | 39,978 | 27% |
| 1K_120L_20W_C4_Device2 | 13.7 | 32,129 | 37% |
| 1K_120L_30W_C1 | 12.3 | 38,913 | 31% |
| 1K_120L_30W_C2 | 13.1 | 24,304 | 49% |
| 1K_120L_30W_C3 | 11.2 | 25,937 | 31% |
| 1K_120L_30W_C4 | 8.4 | 24,228 | 37% |
| 300_120L_20W_C1_Device3 | 14.7 | 19,411 | 57% |
| 300_120L_20W_C2_Device3 | 13.2 | 18,259 | 53% |
| 300_120L_20W_C3_Device3 | 16.8 | 582 | 10% |
| 300_120L_20W_C4_Device3 | 9.1 | 23,449 | 63% |
| 300_120L_20W_C1_Device4 | 17.5 | 50,728 | 32% |
| 300_120L_20W_C2_Device4 | 21.8 | 46,342 | 34% |
| 300_120L_20W_C3_Device4 | 17.2 | 52,345 | 37% |
| 300_120L_20W_C4_Device4 | 14.9 | 38,366 | 38% |
| 300_30L_10W_C1 | 47.5 | 28,544 | 54% |
| 300_30L_10W_C2 | 29.4 | 29,504 | 36% |
| 300_30L_10W_C3 | 15.7 | 21,457 | 28% |
| 300_30L_10W_C4 | 20.7 | 34,950 | 35% |
| 100_120L_20W_C1_Device5 | 8 | 17,348 | 46% |
| 100_120L_20W_C2_Device5 | 9.4 | 19,380 | 49% |
| 100_120L_20W_C3_Device5 | 7 | 17,687 | 38% |
| 100_120L_20W_C4_Device5 | 5.2 | 12,009 | 39% |
| 100_120L_20W_C1_Device6 | 8.2 | 8,612 | 53% |
| 100_120L_20W_C2_Device6 | 10.4 | 13,907 | 53% |
| 100_120L_20W_C3_Device6 | 9.3 | 9,553 | 42% |
| 100_120L_20W_C4_Device6 | 31.3 | 10,326 | 86% |
| 50_120L_20W_C1 | 16 | 4,243 | 48% |
| 50_120L_20W_C2 | 14.5 | 4,904 | 66% |
| 50_120L_20W_C3 | 9.8 | 5,278 | 44% |
| 50_120L_20W_C4 | 9.1 | 12,916 | 39% |

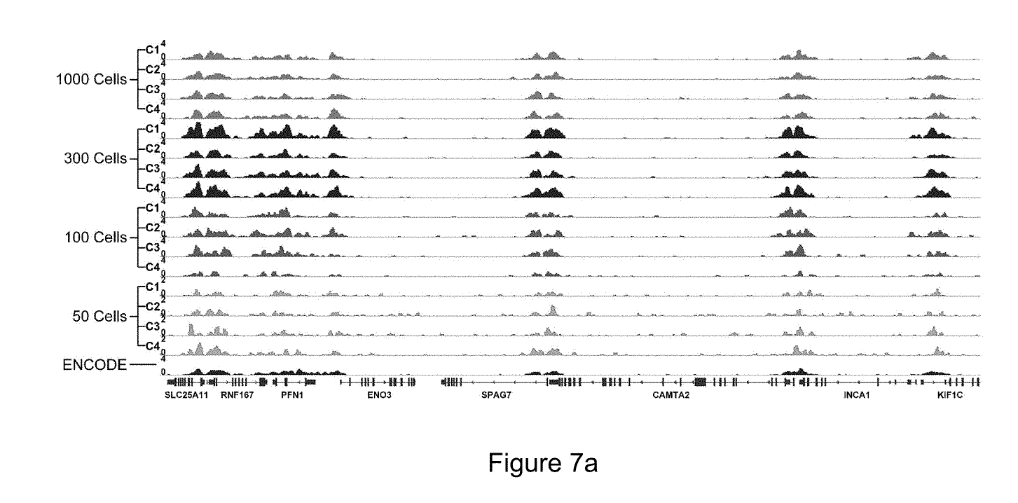

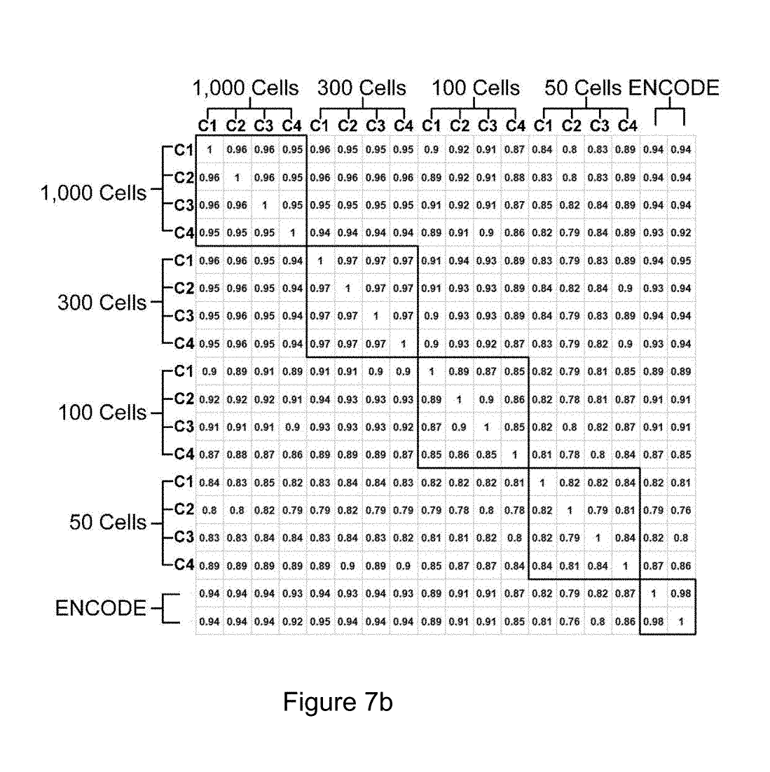

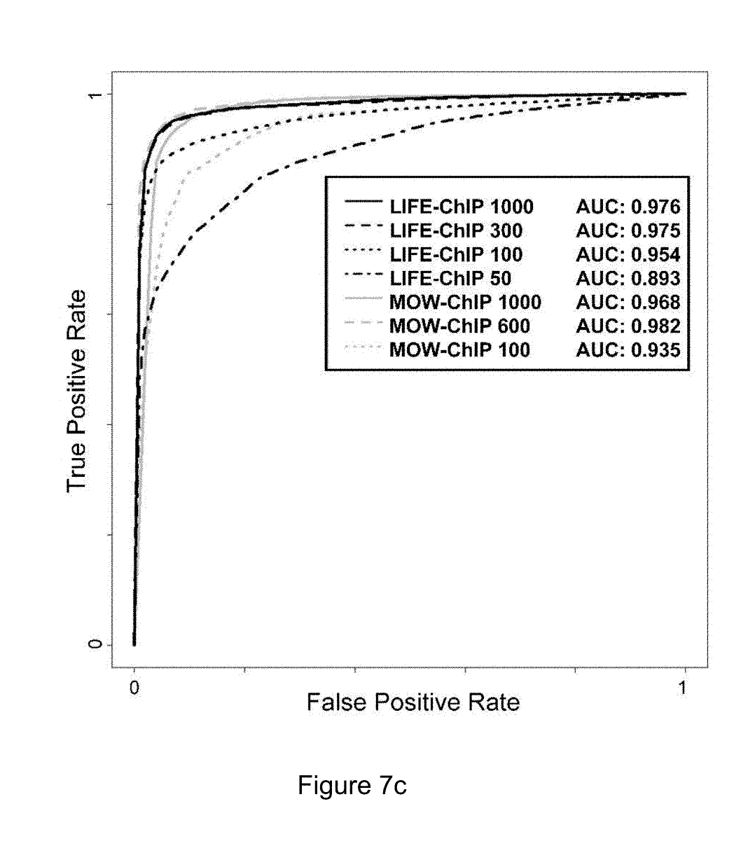

Samples were tested containing various numbers of cells 4000, 1200, 400, and 200 cells (equivalent to 1000, 300, 100, and 50 cells per ChIP-seq assay) (FIG. 7a). The average Pearson correlation coefficients among the data generated by 4 units (c1-4) were 0.958, 0.970, 0.870, 0.820 respectively, while the average correlation coefficients with ENCODE data were 0.936, 0.938, 0.892, and 0.817 respectively (FIG. 7b), showing the ability to produce data with similar reproducibility to published ENCODE data sets (generated using tens of millions of cells), which had an average correlation of 0.937 between replicates. The LIFE-ChIP-seq data quality was also compared to those generated by MOWChIP-seq, which utilized a packed bed of beads combined with oscillatory washing (FIG. 7c). LIFE-ChIP-seq consistently performed better than MOWChIP-seq when similar number of cells were used, in terms of the gold-standard peaks covered.

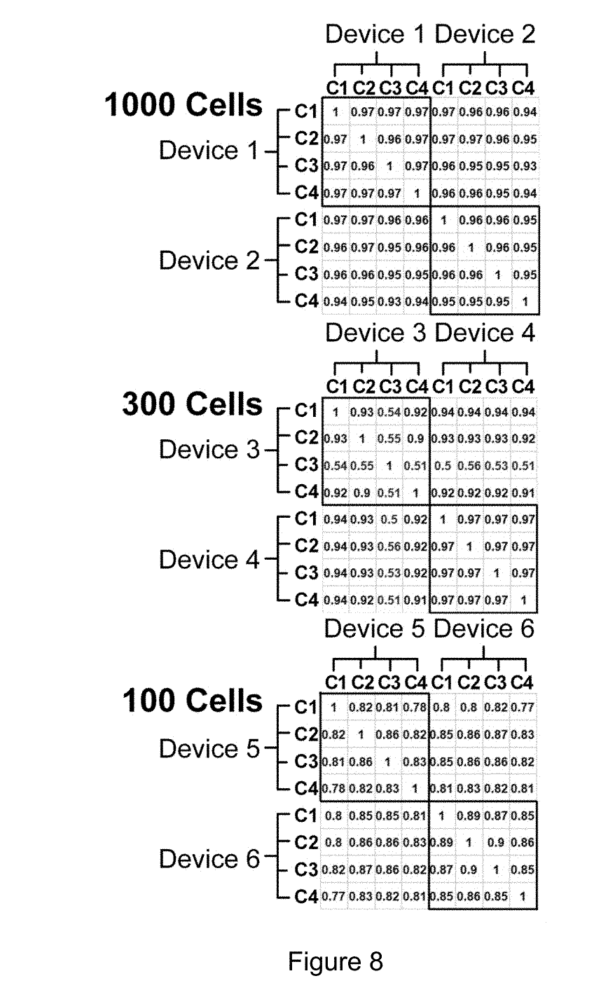

With a parallel system, it is also important to examine reproducibility across different devices. FIG. 8 compared the data collected using two separate but identical devices (each with 4 units) with 1000, 300, and 100 cells per ChIP assay. The correlations were very similar between different units across the 2 devices, with lower cell numbers resulting in lower correlations between replicates. Occasionally there was one failed dataset (e.g. C3 of device 3). However, the device exhibited resistance to cross-contamination, and the other 3 datasets on the same device were not affected.

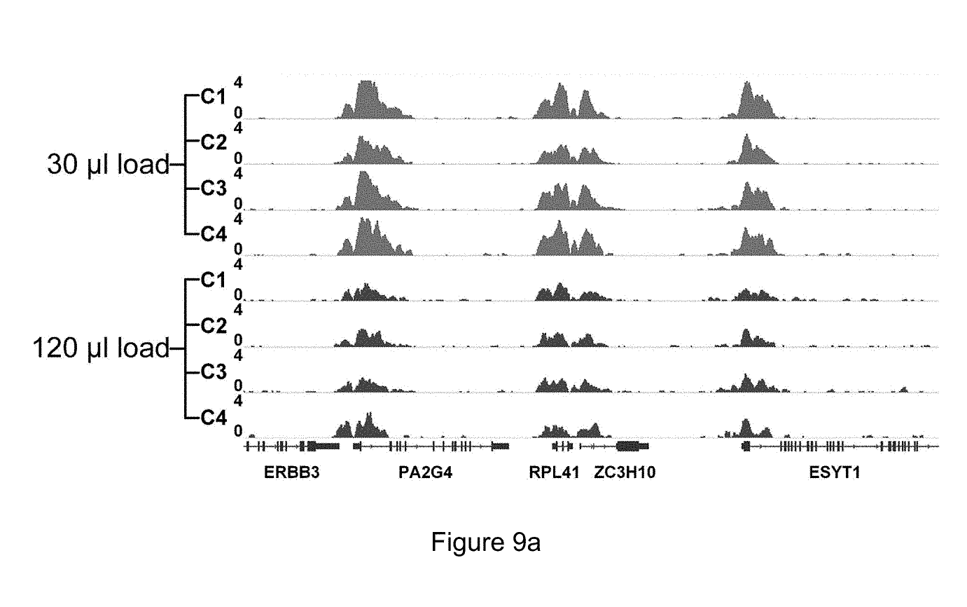

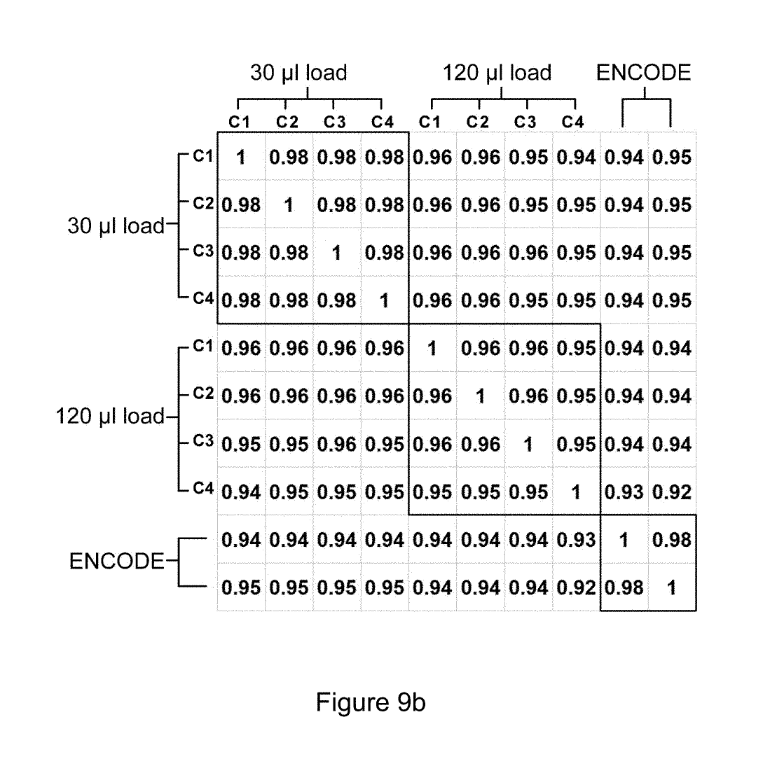

The LIFE-ChIP-seq platform operation was optimized on a number of different parameters. The input volume was optimized at the flow rate of 1 μl/min, which was selected from the fluidized bed behavior described in FIGS. 4a to 4d. Thus larger input volume required longer loading time. The two conditions of 30 or 120 μl input volumes were created by dissolving chromatin from 4,000 cells to either 30 or 120 μl respectively, while keeping all the other operational conditions identical. Generally speaking, lower chromatin concentration (associated with larger input volume) may lead to slower adsorption, depending on the adsorption kinetics. In the results (FIGS. 9a and 9b), it's observed that 30 μl samples indeed had a slightly higher average correlation 0.983 among the 4 replicates produced in one run on the device than that of 120 μl samples 0.956. 30 μl samples also had a slightly higher correlation with ENCODE data than 120 μl samples (0.944 Vs. 0.936). The 30 minute loading condition can be seen to have a higher correlation between chambers in the unit than the 120 minute loading.

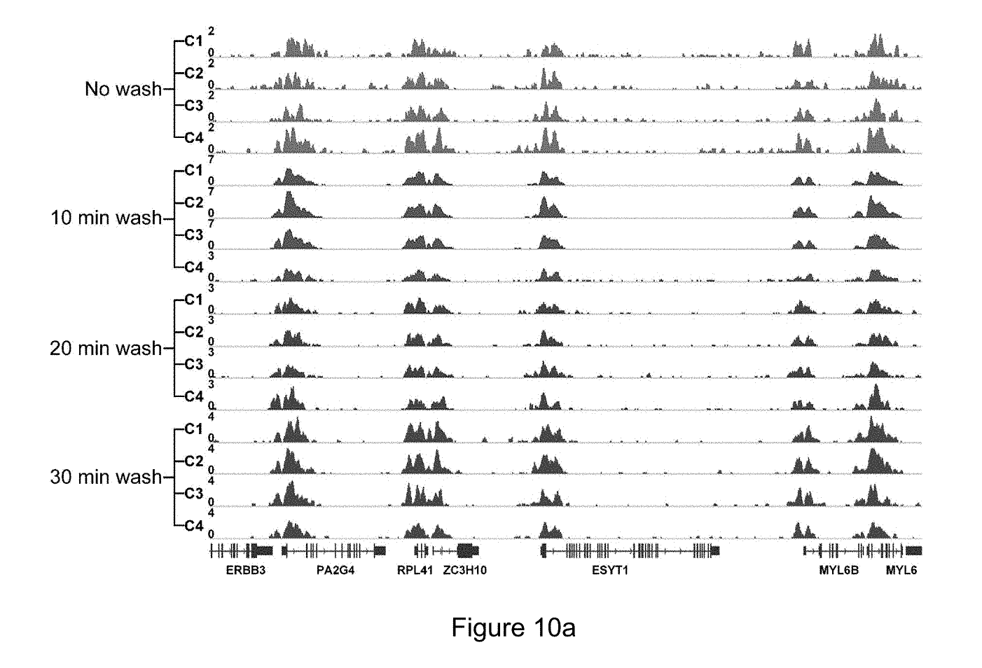

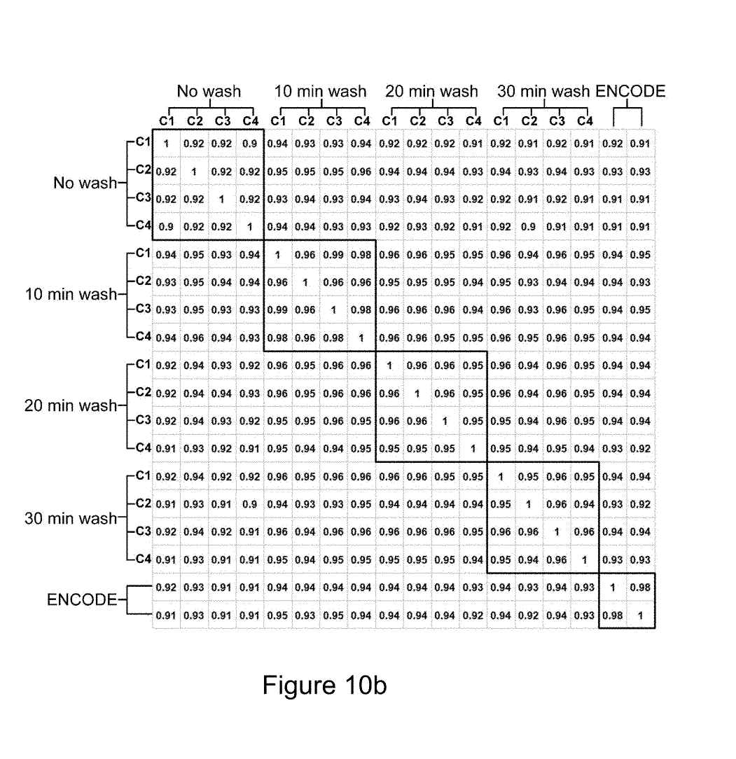

Washing time was another optimized parameter. Washing times were explored varying between 0 and 30 min with 1000 cells per assay (unit). Our LIFE-ChIP-seq data showed that no washing yielded lower-quality data (with average correlation among units of 0.917) which, nevertheless, yielded an average correlation of 0.915 with ENCODE data (FIGS. 10a and 10b). In contrast washing times from 10 to 30 min all yielded high-quality data with only a slight decrease in average correlation among units (from 0.974 at 10 min to 0.956 at 20 min and 0.954 at 30 min) and their correlations with ENCODE data (0.942, 0.936, 0.934 for 10, 20, 30 min, respectively) (FIGS. 10a and 10b). The similarities between data obtained with washing times 10-30 min suggested that the fluidized bed washing was effective for removing nonspecific chromatin molecules.

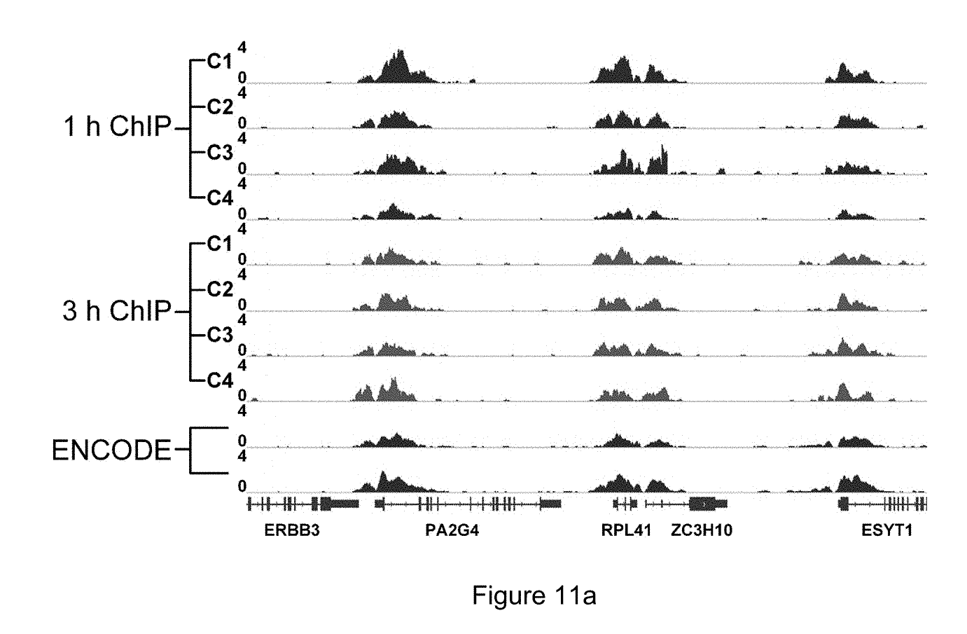

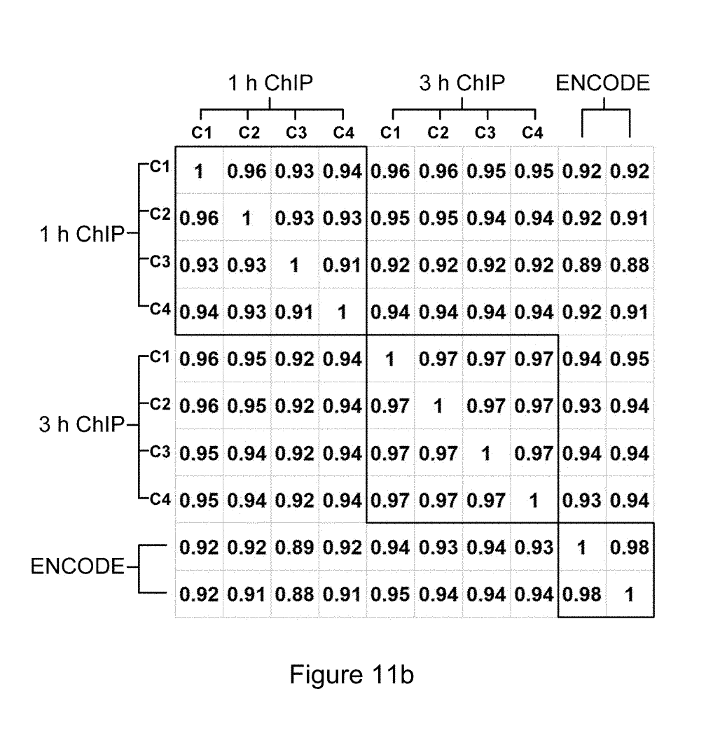

Finally, how the overall time of the LIFE-ChIP assay affected the results was also examined. Using the minimal conditions determined through the optimizations described above, a LIFE-ChIP-seq experiment was performed using 30 min of loading and 10 min of washing per buffer, which equated to a 1 h on-chip assay compared to the 3 h assay (2 h loading and 20 min per washing step) that was set as a baseline. The 1 h assay showed a slightly higher average inter-unit correlation than the 3 h assay (0.964 vs. 0.956) (FIGS. 11a and 11b), but a slightly lower average correlation coefficient with ENCODE data (0.926 vs. 0.938). Thus both conditions produce similar high-quality ChIP-seq data.

### Example 2. Profiling Two Histone Marks in a Single Run

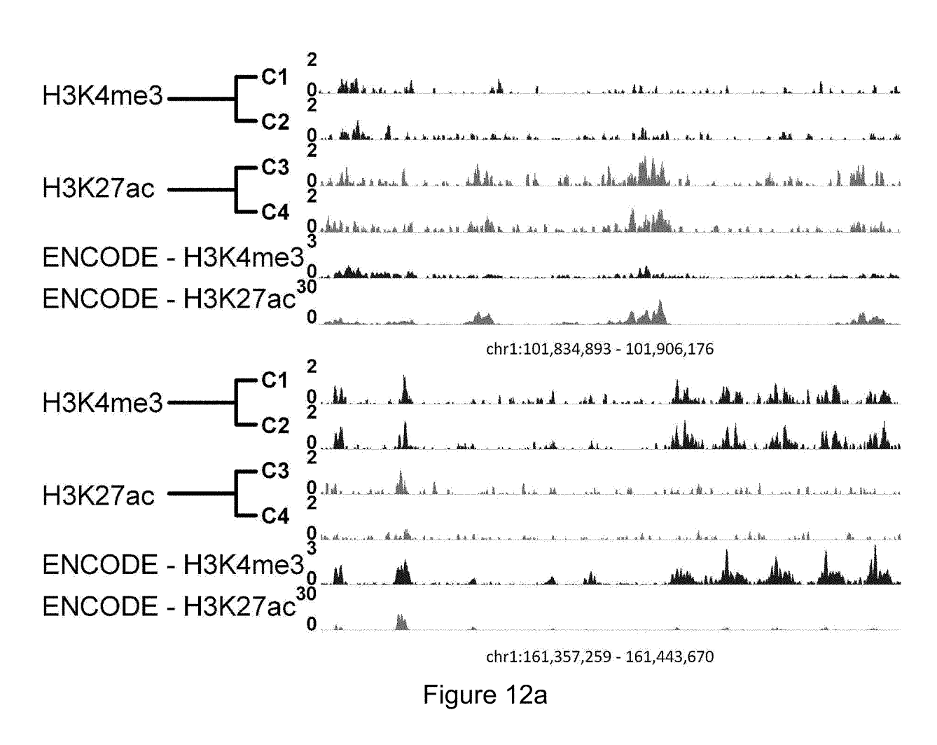

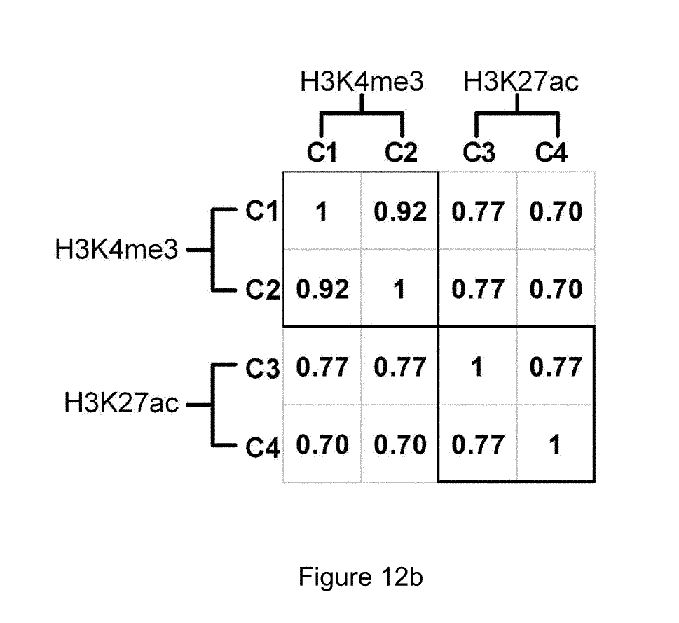

Finally, using a single device, two histone marks (H3K4me3 and H3K27ac) were profiled in one run. Two types of antibody-coated beads (one coated with anti-H3K4me3 and the other with anti-H3K27ac) from two separate inlets were loaded sequentially while avoiding cross contamination (FIG. 5). Loaded beads were directed into different chambers by manipulating the micro-mechanical valves (FIG. 5). A parallel ChIP-seq run was then performed that generated data sets on both H3K4me3 and H3K27ac, each with two replicates, using 300 cells per assay, 120 μL loading volume, and 20 min of washing per buffer. The results of the dual-bead experiment are summarized in FIGS. 12a and 12b. FIG. 12a shows different features in H3K4me3 and H3K27ac at two loci. The data revealed the same characteristics of the two marks that were also shown in ENCODE data. In general, H3K4me3 data sets (with a Pearson correlation of 0.921 between replicates) showed higher quality than H3K27ac ones (with a Pearson correlation of 0.774 between replicates) (FIG. 12b). Nevertheless, H3K4me3 replicates presented much higher correlation between themselves than with any of H3K27ac data sets. This example run demonstrates producing 4 replicates from one sample and profiling 2 histone marks with 2 replicates for each. The parallel operation decreased the time averaged on each assay.

While the invention has been described in terms of exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A microfluidic device, comprising
a plurality of chambers configured to accommodate fluidized beds for chromatin immunoprecipitation assays;
one or more inlet ports configured or configurable to be in fluid communication with the plurality of chambers;
one or more outlet ports configured or configurable to be in fluid communication with the plurality of chambers;
a plurality of micromechanical valves actuatable in different combinations to control and change which of the plurality of chambers are in fluid communication with the one or more inlet and outlet ports and which of the plurality of chambers are not in fluid communication with the one or more inlet and outlet ports at different stages of operation; and
a magnetic field generating device for generating a magnetic field in the plurality of chambers for manipulating magnetic beads.

2. The microfluidic device of claim 1, further comprising a controller configured to run multiple parallel chromatin immunoprecipitation assays simultaneously utilizing fluidized beds in the plurality of chambers.

3. The microfluidic device of claim 2, wherein the controller is configured to run the multiple parallel chromatin immunoprecipitation assays by
loading the plurality of chambers with immunomagnetic beads;
flowing chromatin molecules from one of the inlet ports into fluidized beds containing the immunomagnetic beads to form chromatin-bead conjugates; and
washing the chromatin-bead conjugates with a plurality of sequential wash buffers flowed through the fluidized beds.

4. The microfluidic device of claim 1, wherein the plurality of chambers comprise a plurality of microscale pillars configured to prevent collapse.

5. The microfluidic device of claim 1, wherein the chambers are bell-shaped.

6. The microfluidic device of claim 1, wherein the magnetic field generating device is a permanent magnet or an inductor.

7. A method of performing chromatin immunoprecipitation for multiple parallel assays, comprising
loading a plurality of parallel units with antibody-coated immunomagnetic beads;
flowing chromatin molecules from an inlet into fluidized beds containing the immunomagnetic beads to form chromatin-bead conjugates;
washing the chromatin-bead conjugates with a plurality of sequential wash buffers flowed through the fluidized beds; and
collecting washed chromatin-bead conjugates for DNA release.

8. The method of claim 7, wherein the loading and washing steps are applied in rotating schemes to maintain distribution of reagents among chambers and fluidization of the beads.

9. The method of claim 7, further comprising a step of preparing the chromatin molecules from 100 or fewer cells per assay.

10. The method of claim 9, further comprising a step of preparing the chromatin from 50 or fewer cells per assay.

11. The method of claim 7, wherein the input volume is 5 to 120 μl.

12. The method of claim 7, wherein a total time from a start of the flowing step to a start of the washing step is 10 to 180 minutes.

13. The method of claim 12, wherein the total time from the start of the flowing step to the start of the washing step is 30 to 60 minutes.

14. The method of claim 7, wherein total washing time of the washing step is 0 to 120 minutes.

15. The method of claim 7, wherein a total time from a start of the flowing step to an end of the washing step is 60 minutes or less.

16. The method of claim 7, wherein the collecting step is performed on the microfluidic device.

17. The method of claim 7, wherein the loading step comprises loading different types of antibody-coated immunomagnetic beads into different units of the plurality of parallel units.
